(19) United States
(12) Patent Application Publication
(10) Pub. No.: US 2015/0342906 A1
(43) Pub. Date: Dec. 3, 2015

(54) Anti-sneeze and nose vestibule care - compositions, methods, and devices

(71) Applicant: Yonghua Li, Urbana, IL (US)

(72) Inventor: Yonghua Li, Urbana, IL (US)

(21) Appl. No.: 14/287,266

(22) Filed: May 27, 2014

(51) Int. Cl. A61K 31/137; A61M 35/00; A61K 36/752; A61K 47/10; A61K 31/138; A61K 9/06; A61K 31/19; A61K 31/375; A61K 36/886; A61K 9/00; A61K 36/534; A61F 13/40; A61K 31/495

(52) U.S. Cl. CPC A61K 31/137; A61M 35/006; A61M 35/003; A61K 36/752; A61K 47/10; A61K 31/138; A61K 31/495; A61K 31/19; A61K 31/375; A61K 36/886; A61K 9/0043; A61K 36/534; A61K 9/06; A61M 2210/0618

(57) ABSTRACT

Compositions, methods, and devices are herein disclosed for anti-sneeze and nose vestibule care for humans and other animals. An anti-sneeze composition includes an adrenergic agent, an anticholinergic agent, an antihistamine, a plasticizer for nose skin/nose muscle, and a soothing agent. A version of nose vestibule care composition includes alternative functional agents, such as anti-inflammatory agents, anti-oxidants, and antimicrobials, besides adrenergic, anticholinergic, and antihistamine agents. The alternative or additional agents are present in the invented composition for the purposes of wellness care of noses, prevention of diseases or aiding a medical treatment. The methods and devices for delivering the compositions to the nose vestibules for anti-sneeze, for wellness care, preventive care measure against diseases, and for aiding medical treatment of diseases are prescribed.

### REFERENCES CITED

### U.S. Patent Documents

| | | |
|---|---|---|
| 5,134,166 | July 1992 | Bernstein |
| 5,674,481 | October 1997 | Wahi |
| 5,869,479 | February 1999 | Kreutner, et al. |
| 5,948,414 | September 1999 | Wiersma |
| 5,972,327 | October 1999 | Lin, et al. |
| 6,004,560 | December 1999 | Hsu, et al. |
| 6,086,914 | July 2000 | Weinstein, et al. |
| 6,255,294 | July 2001 | Armstrong, et al. |
| 6,509,492 | January 2003 | Venkataraman |
| 6,599,913 | July 2003 | Johnson, et al. |
| 6,919,348 | July 2005 | Wei |
| 7,013,889 | March 2006 | Cronk, et al. |
| 7,378,082 | May 2008 | Krishnamoorthy |
| 7,582,297 | September 2009 | Reed |
| 7,867,508 | January 2011 | Smith |
| 7,867,523 | January 2011 | Vanterpool |
| 8,158,154 | April 2012 | Chaudry |
| 8,398,580 | March 2013 | Rasor, et al. |
| 8,435,568 | May 2013 | Brosz, et al. |
| 8,450,339 | May 2013 | Moesgaard, et al. |
| 8,486,923 | July 2013 | Marx, et al. |
| 2013/0216574 | August 2013 | Liu |
| 8,575,140 | November 2013 | Wandzel |
| 8,592,381 | November 2013 | Leighton |
| 8,642,069 | February 2014 | Goldin |

### OTHER REFERENCES

Nonaka S, Unno T, Obta Y, Mori S (March 1990) “Sneeze-evoking region within the brainstem” Brain Res. 511 (2); pp. 265-70.
Cole E C, Cook C E (August 1998) “Characterization of infectious aerosols in health care facilities: an aid to effective engineering controls and preventive strategies” Am J Infect Control 26 (4); pp. 453-64.

### DESCRIPTION

### Field of the Invention

This invention relates generally to compositions, methods, and devices for stopping, reducing, controlling, and management of sneezes. The invention also relates to nose vestibule care compositions, methods, and devices. When a subject is a human being, the invention is in personal care arena. It also extends to animal care arena when a subject is an animal such as a horse, a dog, or a cat, etc.

### BACKGROUND OF THE INVENTION

### 1. Sneezes and Concerns

Sneezes, or stemutation, are usually due to the nasal mucosa irritation by foreign particles, also possibly linked to sudden exposure to bright light, temperature change, cold/hot air, a full stomach, or viral infection. Sneezing is also triggered by sinus nerve stimulation caused by nasal congestion and allergies.

While generally harmless, sneezes of unhealthy individuals spread disease through infectious aerosol droplets, commonly ranging from 0.5 to 5 μm. 40,000 droplets can be produced by a sneeze. A sneeze expels air with mucus containing foreign particles or irritants from the mouth and nose in an explosive, spasmodic involuntary action. The release of air and bio-particles from the nasal secretion can reach up to 6 feet distance. Those droplets from sneezes can last on surfaces for 2 to 8 hours. The virus gets into a respiratory system either directly when the droplets reach the eyes, nose or mouth, or when hands touch faces.

The sound of sneezing is a disturbance to quietness as well.

In practice, when sneezing, people cover their mouths by their hands, or turn their heads and bodies to an indifferent or unconcerned direction or place to avoid influencing others, reducing transmission, or contaminating concerned objects. Use of either napkins, or handkerchiefs, or sanitizers, or soaps ensues accordingly.

Devices, like face masks, eye goggles, or face shields/guards, are recommendable and useful at users' preferences, for limiting sneezing influence, or for protection from sneeze.

A conventional preventive technique is a deep exhalation of the air in the lungs, holding the breath for a while, and gently pinching the bridge of the nose for several seconds. This technique works sometimes when individuals know a sneeze coming.

Practices to reduce sneezing in general include avoiding, minimizing or eliminating irritants, pathogens, and allergens.

Also, there is a type of nasal strips that is advertised being anti-sneeze. The strips are worn over users' nose bridges, physically dilating nasal passages for freer breathing.

When sneezes are associated with respiratory diseases, medicines and apparatus for delivering medicines are available for use to treat the diseases. When symptoms of diseases abate, associated sneezes disappear, or diminish to a level of less or no concern as a medical condition. In general, the medical treatments are after the fact measures for remediation and correction.

There is a need of a composition, method, and device that is available to general public for anti-sneezing that is preventive, safe, effective, comfortable, inexpensive, and convenient to use anytime, anywhere, and without concerns to side effects, antibiotics, or systematic overuse of drugs.

Places and situations at which one needs to prevent, stop, control, and manage sneezes are numerous:

Public places: malls, libraries, hospitals, medical care facilities, shelters for homeless and others needed, prisons, museums, buses, trains, passenger planes, bus station waiting halls, airport waiting halls, conference centers and meeting rooms, classrooms, entertainment centers, restaurants, etc.

Situations: in meetings, at a food table, in secret missions, in intimate gatherings, in news reporting, in discussion sessions, in lectures, in preaching and congregations, in surgical and patient care, in food and pharmaceutical production line, in flu season, in allergy season, and during smog invasions, etc.

The lists can be very long. It is an illustration that the need is real and not yet satisfied by a commercial product or a known art.

### 2. Known Arts in Medicine and Medical Treatment of Respiratory Diseases

Many medicines, methods, and apparatus/devices have been invented and developed over time for treating respiratory diseases.

U.S. Patent Application No. 20130216574 (filed Feb. 20, 2012) discloses a kit for providing a combinational therapeutic product comprising a medical device and a pharmaceutical composition to treat mammal diseases. The medical device is for cleansing out harmful substances, such as viruses, multiple drug resistant bacteria, fungi, pollen, dusts, or excessive mucus. The pharmaceutical composition adds the functions of treating medical conditions of respiratory diseases.

U.S. Pat. No. 8,592,381 (filed Jun. 14, 2011) discloses a method for treating rhinitis and sinusitis by rhamnolipids. Rhamnolipids are naturally occurring biosurfactants.

U.S. Pat. No. 8,642,069 (filed Aug. 27, 2009) discloses a composition and method for treating colds. An effective amount of one or more steroids and an effective amount of one or more antihistamines are administered with nasal spray for treating viral infections.

U.S. Pat. No. 8,575,140 (filed Mar. 7, 2011) discloses a composition and methods for treatment of congestion using steroids and adrenergics.

U.S. Pat. No. 8,486,923 (Dec. 20, 2010) discloses use of the combination of ciclesonide and antihistamines for the treatment of allergic rhinitis. Ciclesonide is a steroid.

U.S. Pat. No. 8,450,339 (filed Dec. 11, 2009) discloses compositions for treatment of common cold comprising the combination of a topically active vasoconstrictor and a topically active anticholinergic drug.

U.S. Pat. No. 8,435,568 (filed Jul. 27, 2012) discloses use of WF10 for treating allergic asthma, allergic rhinitis and atopic dermatitis. The method comprises of administering intravenously to the subject a therapeutically effective amount of a pharmaceutical composition comprising chlorite, chloride, chlorate and/or sulfate ions, wherein the treatment is effected not more than once every 6 months, once every year, or once every two years.

U.S. Pat. No. 8,398,580 (filed Dec. 30, 2009) discloses methods and apparatus for treating rhinitis. Devices and methods are described for the delivery of treatment agents simultaneously with capnic gases, to a mucosal membrane at a controlled flow rate.

U.S. Pat. No. 8,158,154 (filed Oct. 31, 2007) and U.S. Pat. No. 7,972,627 (filed Oct. 14, 2005) disclose nasal pharmaceutical formulations and methods that comprise a drug substance (specifically corticosteroids) having a specific particle size distribution profile for improved bioavailability in inhalation.

U.S. Pat. No. 7,867,523 (filed Jul. 18, 2008) discloses a pharmaceutical composition for the treatment of colds and influenza, a mixture of acetaminophen, diphenhydramine, dextromethorphan, arabinogalactan, vitamin C, zinc, olive leaf extract, resveratrol and elderberry extract, for oral administration.

U.S. Pat. No. 7,867,508 (filed Jul. 27, 2007) discloses compositions and methods useful for the non-addictive treatment and prevention of upper respiratory conditions in man and animals, e.g., allergic, non-allergic and mixed rhinitis in man or pharyngitis and IAD in horses. Compositions of the invention comprise effective amounts of a suitable nasal decongestant; a suitable corticosteroid; and a suitable anticholinergic agent. The compositions and methods are for long term use in patients with mixed rhinitis and substantially reduce or eliminate the risk of rhinitis medicamentosa and the risk of adverse sequella seen from therapeutic regimens which employ systemic use of decongestants. The intended application is nasal spray or misting.

U.S. Pat. No. 7,582,297 (filed Apr. 12, 2004) discloses a method of treating or ameliorating a respiratory infection, or a symptom, comprising of administering to a human subject an effective amount of an antibody or fragment thereof that immunospecifically binds IL-9.

U.S. Pat. No. 7,378,082 (filed Nov. 5, 2007) discloses a method for effectively treating allergic rhinitis by delivering a small volume of the epinastine formulation to the nose of a patient using a small volume metered-dose nasal spray pump, without adverse effect of bitter taste. Epinastine is a second generation of antihistamine.

U.S. Pat. No. 7,013,889 (filed Sep. 30, 2003) discloses adhesively applied external nasal strips and dilators containing medications and fragrances so as to deliver aromatic substance through inhalation.

U.S. Pat. No. 6,919,348 (filed May 2, 2002) discloses a method of treating pruritus in a mammalian patient comprising of topically administering a therapeutically effective amount of a cold receptor agonist to skin of the patient. Cold receptor agonist administered further comprises an agent selected from the group consisting of an anti-inflammatory glucocorticosteroid, a local anesthetic, an anti-histamine, a local anesthetic, menthol or a menthol analog and mixtures thereof. The cold receptor agonist is incorporated into an occlusive bandage.

U.S. Pat. No. 6,599,913 (filed Jun. 21, 2002) and U.S. Pat. No. 6,432,972 (filed Feb. 5, 2001) discloses a method of treating and/or preventing allergic and inflammatory conditions of the skin or airway passages in a human comprising of administering orally an effective amount of a nonsedating antihistamine (specifically desloratadine).

U.S. Pat. No. 6,509,492 (filed Sep. 14, 2001) discloses a liquid suspension for treating upper respiratory indications comprising of pseudoephedrine tannate, chlorpheniramine tannate, and dextromethorphan tannate.

U.S. Pat. No. 6,255,294 (filed Dec. 28, 1999) discloses cyanocobalamin (vitamin BI 2) treatment in allergic disease, comprising of delivery to the nasal mucosa of the human by means of one or more nasal drops, sprays, and salves.

U.S. Pat. No. 6,086,914 (filed Mar. 12, 1999) discloses nonsedating formulations for oral administration for allergic rhinitis which possess antihistaminic and anticholinergic activity.

U.S. Pat. No. 6,004,560 (filed Nov. 16, 1998) discloses a nasal spray (drop) for treating fever/cold, by obtaining a liquid by boiling Bupleurum Scorzonerifolium Willd, Radix Lastidis Lndigotica and Folium Lastidis for a long time in water, and then mixing the liquid thus obtained with Vitamin C.

U.S. Pat. No. 5,972,327 (filed Oct. 22, 1997) discloses a method for treating allergic rhinitis in a patient which comprises applying an anti-allergic rhinitis effective amount of a steroid in ointment or cream carrier to the lining of the vestibules of the patient.

U.S. Pat. No. 5,948,414 (filed Mar. 24, 1998) discloses herbal based nasal spray. The composition comprises of herbal-based decongestant, antihistamine, and a saponin. A method is stipulated for treating nasal congestion.

U.S. Pat. No. 5,869,479 (filed Aug. 14, 1997) discloses a treatment of upper airway allergic responses by mixed histamine receptor antagonists with oral or intravenous administration.

U.S. Pat. No. 5,134,166 (filed Oct. 9, 1990) and U.S. Pat. No. 5,008,289 (filed Dec. 2, 1988) disclose compositions and methods for treating the symptoms of certain allergy-related conditions using capsaicin in solution or suspension combined with a selected local anesthetic, topical steroid or antihistamine. Capsaicin is a chemical isolated from the fruits of plants of the nightshade family, principally the pepper plant. Ground pepper, containing capsaicin, has long been utilized by magicians and tricksters as “sneezing powder”, causing intense irritation, producing vigorous sneezing and coughing and, if exposure is extreme, shortness of breath. It has been discovered that capsaicin, when applied repeatedly locally to the nasal mucosa can produce quite contrary effects in treating the symptoms of vasomotor rhinitis, commonly encountered throughout the year, and allergic rhinitis encountered during the ragweed and mold season.

All above arts are in the arena of medical treatments. Requirements of the procedure, dosage, frequency, and duration are to be met for the purpose of a medical treatment.

There are issues with medical agents for treatments of respiratory diseases. Adrenergic drugs, if taken orally or intravenously, could have side effects of blood pressure influence, sedation, altered thought process, depression, and orthostatic or exercise hypotension, altered sexual desire.

If used in nasal spray or mist or inhaler or nasal cleanser or a nasal pump or other type nasal applicator, to effect on nasal mucosa, it could cause medicamentosa or sequella from extended use or systematic overuse. Anticholinergic agents, if taken orally or intravenously, could have side effects of physical and mental impairment. Antihistamine agents, if taken orally, could cause side effects, such as dry mouth, drowsiness (especially with the first generation of antihistamines), dizziness, nausea and vomiting, restlessness or moodiness (in some children), trouble urinating or not being able to urinate, blurred vision, and confusion. Known side effects of corticosteroids, if taken orally or intravenously, could include altered response to physical stress, steroid withdrawal syndrome, weakened immune system, gastrointestinal ulcers or bleeding, osteoporosis, weight gain, insomnia, mood changes, fluid retention and elevated blood pressure, elevated blood sugar, eye problems, atherosclerosis (hardening of the arteries), and aseptic necrosis. There is concern worldwide that antibiotics are being overused. Antibiotic overuse contributes towards resistant infections to antibacterial medications. Specific drugs would have different side effects and degree of side effects depend on dosages, duration and frequency of use. Packages for both prescribed and over the counter medicines have warning labels for children, pregnant women, and people with specific conditions. Medicines and medical treatments are not for use on healthy individuals, extended use, or over use.

There are known general nose/respiratory care products and arts. Saline nasal spray and gel, as well as aromatic inhaler, are intended to provide temporary comfort to some disease symptoms, such as dryness relief and psychological relief. They could be used by a healthy subject for nasal dryness prevention and psychological enhancement of comfort if one wants to use them.

The aforementioned U.S. Pat. No. 7,013,889 (filed Sep. 30, 2003) is intended for treating and releasing congestion and associated with a medical condition, but it could be used for general care if physically wearing a nasal strip or dilator is not a limiting factor. External delivery of aromatic substance through inhalation may have limited efficacy as well.

U.S. Pat. No. 5,674,481 (filed Nov. 20, 1995) discloses an electrostatically charged nasal topical application product for restricting the flow of airborne contaminants into a nasal passage. This would be in a preventive and personal care product arena if the product effects as intended.

The nose is a door to the respiratory system, always open to a breathing environment that contains dust, irritants, allergens, pathogens, and possibly obnoxious gases and toxic chemicals as well. Hence, there is a need of a composition, method, and device that is available to the general public for general nose health care, for respiratory disease prevention, for care of a troubled respiratory nasal system before, between, and after a medical treatment, which is safe, effective, comfortable, inexpensive, convenient to use anytime anywhere, without concerns to side effects, antibodies, or systematic overuse of drugs.

### SUMMARY OF INVENTION

In many of his approaches of attending to and taking care of his sensitive nose, the inventor has discovered a mechanism of effective sneeze prevention, stop, control, and management. Efforts devoted thereafter have established the compositions and methods, as well as devices for delivering the compositions to nose vestibules of a subject for sneeze prevention, stop, control, and management.

The anti-sneeze compositions are safe and effective without concerns to side effects, antibiotics, or systematic overuse of drugs. One essential active agent is an adrenergic agent, that causes vestibule skin and muscle to contract and the contraction propagates to nasal mucosa. The contraction in vestibule skin and muscle, as well as in nasal mucosa, makes the nasal system less susceptible to stimulations from foreign intruders and environmental changes. The other essential active agent is a plasticizer of vestibule skin and muscle. The aforementioned contraction causes rigidity of vestibule skin and muscle. The plasticizer in the composition releases the rigidity. The third active agent is an antihistamine agent that inhibits histamine right in the vestibule. The fourth active agent is an anticholinergic agent that prevents excessive nasal secretion. The fifth element in the composition is a soothing agent, such as a pleasant aromatic substance, which creates a temporal comfort and attractiveness in use.

In his pursuit of means for bettering his nose system, the inventor gained a general improvement of his wellness by the applications of compositions he tried. It is understandable, because the nose is the frontier of the respiratory system and connected to the face muscles and brain functions through neurons. The inventor has gone further and developed compositions, methods, and devices for general nose vestibule care for the purposes of comfort and respiratory disease prevention, and for care of a troubled nasal system before a medical treatment, between medical treatments, and after a medical treatment.

The nose vestibule care compositions are safe and effective without concerns to side effects, antibiotics, or systematic overuse of drugs. In addition to the agents in anti-sneeze compositions, an optional agent herein is an anti-inflammatory agent that is for preventive care of/or reducing infections that are common in the base of the hairs within the vestibule. The second optional agent is an antimicrobial that is for prevention or inhibition of air-born or contact-transmitted microbial (virus, bacterial, fungus, yeast, and parasite) infection in the lining of nose vestibules. The third optional agent is an antioxidant that is for reduction of harmful oxidant molecules from air pollutants in the lining of nose vestibules.

The method as well as delivery is easy, convenient, and friendly to use anywhere and anytime. The aforementioned compositions (for anti-sneeze or for general vestibule care) are embodied in either a pre-medicated swab, or a medicated “nose stick”. The tip of the nose stick is shaped and sized for easily entering nose vestibule. The composition is applied by swirling and massaging the nose stick against vestibule skin.

### DETAILED DESCRIPTION OF THE INVENTION

### Anti-Sneeze Compositions

It is observable that sneezing typically occurs when foreign particles or sufficient external stimulants reach and/or pass through the nasal hairs. It is believed the nasal mucosa is being stimulated That triggers the release of histamines. The nerve cells in the nose are irritated, resulting in signals being sent to the brain to initiate the sneeze through the trigeminal nerve network.

So the mechanism of sneeze prevention, stop, control, and management in this invention is as following: (1) an adrenergic agent applied to nose vestibules makes vestibule skin contract. The contraction propagates to and through nasal mucosa, nasal mucosa becomes less sensitive to stimulation: (2) antihistamine agent applied to nose vestibules acts on histamine receptors, and histamine release is depressed; (3) the plasticizer in the composition tenders the rigidity in vestibule skin and muscle created or elevated by the constriction; (4) anticholinergic agent prevents excessive nasal liquid secretion. In addition, the soothing agent, an aromatic or other, provides a pleasant experience of use.

### Use of Adrenergic Agent for Anti-Sneeze in Present Invention

Medical use of adrenergic agent is well known. These drugs are used to combat acute attacks of bronchial asthma, shock, cardiac arrest, and allergic reactions, as in nasal decongestants and appetite suppressants. Example agents used as nasal decongestants are pseudoephedrine and phenylephrine.

Pseudoephedrine is used in many over-the-counter drugs. There is a concern that it can be used to make methamphetamine, an illegal drug. Its alternatives as decongestants include phenylephrine.

Phenylephrine hydrochloride is a FDA approved drug. Examples approved include up to 10% in ophthalmic solution/drops, and 10 mg/ml in infusion solution. Some over-the-counter cold & flu oral medicine contains 5 mg of phenylephrine hydrochloride in 15 ml of liquid with other functional agents. Over-the-counter oral tablets for decongestion and sinus pressure relief may contain 10 mg of phenylephrine HCl per tablet.

For adults and children over 12 years and over, the oral tablet use direction is one tablet every four hours, not more than six tablets every twenty four hours, and stop use within seven days if symptoms do not improve or a medical complication occurs. So the least amount of oral intake of phenylephrine HCl is 10 mg if only one tablet is taken, and up to 60 mg per day.

In present invention, the application is topical, limiting to vestibule skin. The exemplary application level/effective level is around a range of 0.005 mg to 0.02 mg pick-ups of phenylephrine HCl by the skin in both nose vestibules per application. Compared to 10 mg tablet oral dosage for medical use of decongestion, phenylephrine HCl in anti-sneeze application is thousands of times less in usage.

### Use of Antihistamine Agent for Anti-Sneeze in Present Invention

Medical use of antihistamines for allergy symptoms relief is well known, including suppressing itching and sneezing associated with allergy. It is understood that the second generation of antihistamines are less likely to cause sedation/drowsiness. Examples of both first the generation and second generation of antihistamines are many. The inventor uses cetirizine to illustrate the use of an antihistamine agent as one of the components for anti-sneeze applications of present invention.

Cetirizine hydrochloride is a FDA approved drug. Examples approved include 5 mg/10 mg tablets for oral medication of allergy or hives. Over-the-counter oral tablets relieve allergy-related sneezing, itchy/watery eyes, runny nose, and itchy throat/nose.

For adults and children 6 years and over, the use direction is one tablet daily. Warnings are given in the label for potential complications, and for pregnant or breast-feeding women. A 5 mg product may be appropriate for less severe symptoms.

In present invention, the anti-sneeze composition is a topical application, limiting to vestibule skin. The exemplary application level/effective level is around a range of 0.005 mg to 0.02 mg pick-ups of cetirizine HCl by the skin in both nose vestibules per application. Compared to 10 mg tablet oral dosage for medical use of allergy relief, cetirizine HCl in anti-sneeze application is thousands of times less in usage.

### Use of Plasticizers for Anti-Sneeze in Present Invention

Many well proven skin emollients/moisturizers can be utilized for this purpose, such as glycerin and propylene glycol. These are FDA approved as generally recognized as safe (GRAS) material. They will be safe as components for anti-sneeze in present invention. As an illustration, a composition of anti-sneeze comprises of one part water, one part glycerin, and one part propylene glycol. The mixture also serves as a solvent system for adrenergic agent, anticholinergic agent, and antihistamine agent.

The application with a swab or a nose stick would result in a total skin pick-up in both vestibules around a range of 0.01 g to 0.03 g for an average adult user. As such, the skin pick-up of glycerin or propylene glycol is estimated being around a range of 0.0035 g to 0.01 g for that user. The application with a pre-made swab or a nose stick does not result in any dripping. A wet feel in a vestibule, if the application carries an excess of liquid to the skin, may not be desirable by the user. The plasticizers would be discharged with nose secretion when the user blows his/her nose.

### Use of Anticholinergic Agent for Anti-Sneeze in Present Invention

Inclusion of anticholinergic agent in the compositions is expected to have a potency to prevent excessive nasal secretion. Some of the first generation antihistamines like diphenhydramine have anticholinergic functionality. Non-antihistamine anticholinergic agents are well known as well, like ipratropiumn. An anticholinergic agent blocks the neurotransmitter acetylcholine in the central and the peripheral nervous system. What it does in a nasal system is reduce nasal discharge/running nose.

Diphenhydramine HCl is explained further herein as an illustration of use of an anticholinergic agent in nose wellness care compositions. It is a FDA approved over-the-counter drug. Oral tablets may contain diphenhydramine HCl of 25 mg and 50 mg. It is marketed as allergy relief for sneezing, runny nose, itchy/water eyes, and itchy throat. It is also approved as a sleep aid due to the drug's sedative potency.

For a 25 mg tablet, the instruction of use for one over age 6 is to take one tablet every 4 to 6 hours, not more than 6 times every 24 hours. Other warnings of complications besides sedation are included in the label.

In present invention, a composition of nose wellness care is topical application, limiting to vestibule skin. The exemplary application level/effective level is around a range of 0.0075 mg to 0.05 mg pick-ups of diphenhydramine HCl by the skin in both nose vestibules per application. Compared to 25 mg tablet oral dosage for medical use of allergy relief, diphenhydramine HCl in presently invented application is thousands of times less in usage. By the low level of usage and the topical application to vestibule skin only, the drug's drowsiness and other side effects are not expected. However, individual users may have unique experiences. The drowsiness, if any induced, can be perceived as a benefit for night use or for other specific use. Otherwise, a non-antihistamine nature anticholinergic agent is to be used in a presently invented composition and the amount of use will be thousands of times less than that in a medically effective oral dosage as well.

### Use of a Soothing Agent for Anti-Sneeze Composition of Present Invention

Many soothing agents exist as skincare/cosmetic ingredients, including botanical/herbal extracts. FDA approved aromas, especially those used as food additives, are desirable. Aroma presence in the composition provides a pleasant experience of use, and may also induce a psychological feel of soothing. A specific and strong aroma/fragrance itself, however, may cause sneezing in some people, should be avoided, and an alternative can be used. As an illustration, a drop of coconut extract, a drop of lemon extract, a drop of banana extract, and a drop of mint extract were added into four aforementioned anti-sneeze composition formulae respectively. A group of people tried the formulae on their nose vestibules, one person picked the formula with lemon extract addition as more attractive, and other person picked the formula with coconut extract as more attractive. None of the formulae smelled too strong to cause any irritation. So it will be empirical to determine the type and addition concentration of an aroma to be used for an anti-sneeze composition. It can be a choice by an end user.

### Wellness Nose Care Compositions

The air we are breathing is full of particles, chemicals, and microbes. The exact contents and concentrations vary with locations, weather, and seasons. In flower booming season, distribution and concentration of pollens are concerns of allergy. In cool/cold weathers, cold virus and influenza are sources of respiratory infection. In industrial polluted locations and areas, airborne chemical species invades respiratory systems through open noses. In damp and not well ventilated confinements, houses, and specific workplaces, mold spores abound. As such, general nose care as a personal care resource is indeed a need and desirable, if one knows or anticipates he/she would be in an aforementioned concerned environment/situation. In some instances, masks/face guards will provide protection. In other instances, to have/wear the nose care product of present invention may be a better, more feasible, and/or complimentary alternative.

The composition comprises of an adrenergic agent, an antihistamine agent, an anticholinergic agent, a plasticizer, a soothing agent, and optional agents of following functions: anti-inflammatory, antimicrobial, and antioxidant.

As in aforementioned anti-sneeze composition, an adrenergic agent would constrict vestibule skin, and the constriction would propagate to inner nasal cavity mucosa. The antihistamine agent would inhibit histamine release from the upper respiratory system. The anticholinergic agent would prevent excessive nasal secretion. A plasticizer would release the rigidity associated with the constriction created by the antiadrenergic agent. As a result, the wellness of the nose/respiratory system is served by its reduced susceptibility to irritants and allergens.

### Anti-Inflammatory Agent in a Nose Wellness Care Composition

Small infections are common within the nasal vestibule in the base of the hairs. That underlines the benefits of the presence of an anti-inflammatory agent in the compositions for nose wellness care.

Many ingredients in skincare like allantoin or aloe vera do provide anti-inflammatory function. Inclusion of those ingredients in the nose wellness care compositions is more favorable than using regulated anti-inflammatory drugs, because the composition is intended for individual care without professional medical guidance.

### Antimicrobials in Nose Wellness Care Compositions

It is readily understood that inclusion of antimicrobials in a presently invented nose wellness care composition will be beneficial. Noses are openly accessible to both airborne and contact-transmittable microbes, including bacterial, mold, yeast, and virus. The applied antimicrobials to nose vestibules pose the first stance against the invasion and propagation of pathogens to vestibule skin and respiratory mucosa.

The skincare industry has a history of using antimicrobials in skincare formulations. There are antimicrobials that are FDA approved food additives (benzoates, sorbates, for example). There are also naturally originated substances like plant/herbal extracts (tea tree oil, rosemary oil, eugenol, to name a few) that are safe to use with no observable adverse effects. Under a general guidance of any component in present invention being safe and bearing no adverse effects, combining two or more antimicrobials into the composition is pursuable for a broader spectrum of antimicrobial activities. Amount of addition of a specific antimicrobial in a nose wellness care composition is to follow FDA regulation if any.

The presence of the antimicrobials in the compositions will serve as preservatives to the formulated products as well.

### Antioxidants in Nose Wellness Care Compositions

Benefits of antioxidants in skincare have been advertised extensively. Antioxidants can counter “free radicals” that damage skin cells. A lot of foods and nutritional supplements are rich in natural antioxidants, like vitamins. Acai oil, alpha-lipoic acid, green tea extract, retinol, vitamin C, CoEnzyme Q-10 (CoQ-10), caffeine, alpha-hydroxy acid, salicylic acid, and hyaluronic acid. These ingredients are well known antioxidants.

Inclusion of antioxidants in a nose wellness composition is perceivably rational. Herein vitamin C is used as an illustration. According to past researches, vitamin C does significantly improve immune function but does not prevent the common cold or decrease the severity of symptoms in non-athletic people by oral intake. In present invention, vitamin C is used as an antioxidant against potential radical damage to vestibule skin. By working with all other components of the nose care composition, it can have a better chance to stop or inhibit cold virus right in the vestibule, and so common colds are potentially prevented.

### Method and Devices for Delivery of an Anti-Sneeze Composition or Nose Wellness Care Composition

A composition of anti-sneeze or nose wellness care is to be delivered only to nose vestibule skin area by a tool that can transfer, spread, and massage active agents onto and into the skin.

In essence, any tool, like a finger or a cotton swab, can be used to serve the purpose of aforementioned application. A pre-medicated/pre-assembled/manufactured tool is preferred for adequate and even, but not excessive application, for convenience of storage and carrying, and for elegance of the usage.

An option of such a tool is a swab with an absorbent tip, pre-impregnated with an aforementioned composition. The material for the absorbent tip can be cotton fibers or any other suitable fibers, polymer or matrix that can possess required absorbency. The swab can be sealed individually or in a group package.

A second option of such a tool is a “nose stick” with a cone shaped tip, or a smooth tip of any shape, like a conventional lip stick in a tube. The stick/core of the tube is a stiff waxy compound of one aforementioned composition with filler/extender/binder/base material/materials like carnauba wax. The filler/extender/binder/base material/materials solidify the composition, and make the composite compoundable into the desirable stick shape and hardness. The hardness of the stick is as such that it has enough solidity and tenacity to hold the shape in massaging against the vestibule and yet the tip would transmit and spread the composition to the skin.

The other option of the delivery tool is a “nose stick” tube, similar to a lipstick style tube. The core of the tube, however, is a solid absorbent material matrix, or foam, that contains the liquid of anti-sneeze or nose wellness care. The composition is readily applied to the lining of a nose vestibule when the smoothly shaped tip of the solid absorbent material is pressed, swirled and massaged against the lining. The composition liquid can wick through the absorbent tube to the tip, or flow through to the tip by a pressure applied from the bottom, or by a lateral squeeze. The absorbent core can be formed by polymer fibers tangled around a hard stick (wooden, plastic, or in any other nature), or a polymer web/matrix wrapped around a hard stick, or a foam padded around a hard stick. It is expected the absorbent core is compatible and stable with the retained liquid because the liquid does not have corrosive or reactive element.

The other option of the delivery device is a liquid containing “nose stick” tube. The smoothly shaped tip has capillaries of liquid passage. The contained liquid in the tube comprises the aforementioned anti-sneeze composition/or nose vestibule care composition. When the tube is squeezed or applied with a pressure, the liquid is readily wicked through the capillaries to the surface of the tip, which is pressed, swirled and massaged against the lining of the vestibule to deliver the composition.

Yet another option of the delivery device is a specially designed capped nose stick, of which the cap has a lining of absorbent material or is stuffed with an absorbent material. The anti-sneeze composition or nose wellness care composition is retained in the absorbent material. The absorbent material is fully wetted but not dripping. The tip of the nose stick has an outer layer of absorbent material as well. It is wetted when the stick is capped. After the wet tip being used against nasal vestibules, the tip will be re-wetted by the composition contained in the cap when the stick is encased.

These devices, especially the secondly described optional device, can also serve to stabilizing the composition. The devices limit the aqueous content to a minimum, and limit the composition exposure to air to a minimum, as such increasing the survival potential for formulation ingredients from separation, aqueous hydrolysis and oxidation during storage.

The present invention is illustrated by following examples.

### Example 1

Phenylephrine HCl 12.5 mg was dissolved in a 15 ml liquid solvent. The solvent comprises of one part water, one part glycerin, and one part propylene glycol. One cotton swab weighed 0.120 g. The swab soaked into the phenylephrine solution, was squeezed off excess liquid, and weighed 0.314 g. So the phenylephrine solution pick-up by the swab was 0.194 g. After swirling and massaging against two nose vestibules of a 5.5 feet tall adult subject, the swab weighed 0.295 g. The adult cleared his nose before the application. So his vestibule skin picked up 0.019 g of phenylephrine solution. By calculation, a net 0.016 mg of phenylephrine HCl was applied to his vestibules.

### Example 2

On a September day of 2013, a solution containing phenylephrine HCl (5 mg/15 ml) was applied to the vestibules of a 53 year old adult male by a cotton swab. The subject was a chronic sneezer. He felt almost instant relief, and did not sneeze for quite some time. That was the first time of application of present invention. The detail was not recorded and how long he kept sneeze-less after the application was not monitored. At the application and sometime afterwards, he noticed the slight rigidity in his vestibules.

### Example 3

A solution was made comprising of phenylephrine HCl 10 mg in a 15 ml solvent. The solvent has equal parts of water, glycerin, and propylene glycol. Glycerin and propylene glycol herein were intended as plasticizers for the vestibule skin and muscles. The solution was tried on to the nasal vestibules of two subjects, a male human and a female human, by cotton swabs swirling and massaging against the vestibules. Both subjects reported a relaxed experience in the vestibules by the new composition, compared with previous ones that did not have the plasticizers.

### Example 4

The samples of the solution described in Example 3 were added with variety of food fragrances respectively, such as lemon extract, banana extract, coconut extract, rhum extract, and mint extract. The scented samples were tried by a group of four people on their nasal vestibules. Lemon scented and coconut scented samples were picked up being more soothing or generating more attractive feels in use than others. All users in the group agreed that a soothing or a physical/psychological attractive experience is an important and integral factor in the intended application. There will be preferences among consumers over specific fragrances incorporated.

### Example 5

On a cold day of January 2014, the outside temperature was minus 19 degree Celsius. A 53 year old male subject got up, having heavy head, sneezes and running nose. He applied a solution of present invention of 5 mg phenylephrine to his nasal vestibules. The subject felt no sneezing and was comforted for quite some time in the morning afterwards. That was reminiscent to him that he could apply the technique every time he feels the urge for sneezing.

### Example 6

On a day of middle January 2014, a 53 year old female used the anti-sneeze formula of present invention containing 5 mg phenylephrine. The subject had chronic high blood pressure and a couple of other medical concerns. She would not take phenylephrine, or cetirizine, or diphenhydramine tablets out of precautionary concerns. In the morning, she had a spell of sneezes and itching face. She applied the formula three times in that day with a cotton swab massaging her nose vestibules. By the second day, both her sneezing and itching were relieved. The relief of face itching is explainable by the fact that the nasal muscles and face muscles are connected.

### Example 7

On a day of late January 2014, a 10 year old girl used the anti-sneeze formula as in Example 6. There was an adult in the house who had a spell of sneezes, and the girl caught sneeze one or two. Both of them were relieved of sneezing by applying the formula to their nasal vestibules in the rest of the day.

### Example 8

On a day in the late January 2014, a 15 year old girl had sneezes around 3 pm after gym activity. She was offered to apply the technique of present invention with a solution of phenylephrine 5 mg to her nasal vestibules. She had one or two sneezes between 3 pm to 7 pm. She was advised to blow her nose clearing her nasal secretion and apply the technique again at 7 pm. She was sneeze-less afterwards.

### Example 9

On a day of early February 2014, in the evening, a 15 year old girl had a spell of sneezes. She complained something in the air of the house causing her to sneeze. She was offered to apply the anti-sneeze formula in the same way as in Example 8. She felt her nose cooled down at the application. A few minutes later, her nose had secretion, and she had to blow cleaning it. At that, she doubted the effectiveness of the application. But another few minutes later, she started to feel well, and remained sneeze-less in the rest of the evening and night. It was noted that she did not have any sneeze the second day either.

### Example 10

On a day of middle February 2014, the weather was cold due to a cold spell sweeping the region for the last few days. In a family of four, three people sneezed in the morning, the anti-sneeze formula same as in Example 9 was used with cotton swabs by the three family members. The family remained sneeze-less for the rest of the day.

### Example 11

To illustrate what an adrenergic agent would do to a nasal vestibule in a highly concentrated application, the following experiment was done. From the knowledge and FDA regulation, phenylephrine HCl can be used as 10% ophthalmic solution/drops and 10 mg/ml in infusion solution. Conventional oral tablets for decongestion is 10 mg per tablet. So it is understood that a cotton swab containing 10 mg of phenylephrine HCl (about 5% in water solution), swirling and massaging against the nasal vestibules for a test would pose no risk to the subject. A 53 year-old human male applied the swab on his both vestibules. The effect was that he felt his vestibule skin tighten up for about 15 minutes, and no urge for sneezing in the rest of the day. The experience confirms again the need of a plasticizer and a soothing agent in the composition for the benefit of relaxation of use.

### Example 12

After a couple of times using presently invented method of a solution containing 5 mg phenylephrine HCl in 15 ml solvent, a 53 year-old male, a chronic sneezer, started to believe in the methodology, and to use the application every time he had the urge for sneezes. The application worked every time to relieve him at the time of application. The manner of application was to clear his nose of the nasal secretion first, and then to swab and massage his nasal vestibules. Clearing the nasal secretion was noted important, because holding nasal secretion in the nasal system sometimes causes sneezing. The adrenergic agent causes contraction in the vestibules and propagates to the inner nasal passage. The contraction results in easier nasal secretion discharge. The subject sometimes felt it needed to clear his nasal secretion after an application, repeat the application, and the effect was more pronounced and lasted longer.

### Example 13

An anti-sneeze solution of present invention containing 5 mg of phenylephrine HCl was used by a 53 year old male. After swirling and massaging his nasal vestibules with phenylephrine solution, he massaged the vestibule skin with other swab of glycerin. He noted that the tense feel of rigidity from the contraction caused by the antiadrenergic agent did not arise this time.

### Example 14

A composition of present anti-sneeze invention was made of 12.5 mg of phenylephrine HCl in 15 ml solvent. The solvent contained 7.5 ml of glycerin and one ml of lemon extract. A 53 year old male, a chronic sneezer, applied the composition to his vestibules with swab, swirling and massaging against his nasal vestibules. He felt almost instantly refreshed and relieved from the urge of sneezes, without a feel of vestibule skin constriction. The effect lasted about 3 to 4 hours.

### Example 15

In an afternoon of an early March day of 2014, during traffic rush hours, after taking presently invented anti-sneeze application, a 53 year old male, a chronic sneezer, went out onto the sidewalk of a busy street in town for a jog/walk. There was significant car fume pollution along the way. Surprisingly, he did not sneeze at all in the trip. This was very unusual for him. He would have been frequently irritated by the polluted air and sneezing unpredictably along the way if not taking the anti-sneeze application.

### Example 16

On a day of early March, 2014, a 10 year old girl was offered for the anti-sneeze application. Her school sent in a note that day that there was a case of strep throat in her classroom. It was out of precautious consideration that the anti-sneeze application might make her less susceptible to virus invasion due to her vestibule skin constriction by the adrenergic agent. She did not have any wellness issue in that period.

### Example 17

An anti-sneeze formula was made of 10 mg cetirizine in a 10 ml solvent with equal parts of water, glycerin, and propylene glycol. One ml of banana extract was added as soothing aroma. It did not create a strong appealing smell. So One ml of coconut extract was added. The resulted product had an attractive appeal to the user. The user was a 53 old chronic sneezer. He applied the formula to his nasal vestibules around 10 am. At the time of 10 am, he had the familiar lousy feel in his nose. That became a cue for him to clear his nose and do the anti-sneeze application. He did not have any sneezing urge before 4 pm that day.

### Example 18

On a day of middle March, 2014, a 53 year old female used the anti-sneeze formula of present invention as in Example 12. The subject had chronic high blood pressure and a couple of other medical concerns. She would not take phenylephrine, or cetirizine, or diphenhydramine tablets out of precautionary concerns. In the morning, she had a spell of sneezes, and was offered the anti-sneeze formula. She did not sneeze any more during that day after application of the formula.

### Example 19

On a day of middle March, 2014, a 53 year old male chronic sneezer, and a 53 year old female, both used an anti-sneeze application in the morning before an excursion. The female had a couple of sneezes that morning, and thought that may be due to weather suddenly warming up. The female subject had one more sneeze a few minutes after the application, and was advised to blow clear the nasal secretion. After that, she did not sneeze any during that day. The male, the chronic sneezer, sneezed only once a couple of hours later in the excursion under the warm sunshine in the car. He did not sneeze any more before the late afternoon. That was unusual, because he used to put napkins beside him or with him when he was driving and in excursions for frequently occurring sneezing. The application formula was 10 mg phenylephrine and 10 mg cetirizine in a 20 ml solvent of equal parts of water, glycerin, and propylene glycol. Coconut extract was used as aroma.

### Example 20

In an evening of middle march, 2014, a 53 year old chronic sneezer applied the anti-sneeze formula as in Example 14, before going to a concert. He did not feel any urge to sneeze during the concert. That was unusual. He used to take physical means to depress his urges to sneeze or went out in between to release himself in that kind of hours-long situations.

### Example 21

On a day of mid-March, 2014, a 15 year old girl felt lousy in her nasal system and sneezed. She was offered with the anti-sneeze formula same as in Example 14. After the application, her lousy feel was relieved and did not sneeze afterwards.

About the same time, her mother sneezed as well. The same formula was offered and applied to her vestibules. As expected, she sneezed no more afterwards.

### Example 22

On a day of mid-March, 2014, a 53 year old male, a chronic sneezer, had to take a trip to another city hospital that involved about one hour driving one way. He used the same formula as in Example 14 for his nasal vestibule application around 7:30 am. There was no any lousy feel and no sneezes in his nasal system that he would usually have experienced, in his driving and afterwards hospital visit. He had one sneeze after his lunch, so the application kept him sneeze-less for 5 hours.

### Example 23

An anti-sneeze formula was made of 25 mg diphenhydramine HCl in a 10 ml solvent of equal parts of water, glycerin, and propylene glycol. A mint extract was added as aroma. Diphenhydramine HCl is a first generation antihistamine with strong anticholinergic function. It was tried on by a 53 year old male at the time the formula made to see if the application would induce any drowsiness. On that particular day, he had a slightly unusual feel, and was not sure about his judgment. Two days later, the weather turned snowy and freezing again. In the morning, he had more nasal secretion. The application of the formula same as in Example 14 did not help in depressing the feel for nasal secretion. He used the formula of diphenhydramine, and it indeed relieved his lousy feel and sneezes.

### Example 24

An anti-sneeze formula was made of 10 mg phenylephrine HCl, 10 mg cetirizine HCl, and 25 mg diphenhydramine HCl, in a 30 ml solvent of equal parts of water, glycerin, and propylene glycol. Coconut extract was used as the aroma. A 53 year old male, a chronic sneezer, applied the formula to his nasal vestibules by swab swirling and massaging against his vestibule skin. The relief was felt almost instantly, and the comfort rendered was noted being greater than that from the formulae of phenylephrine and cetirizine and could last up to 6 hours or so. It was reasonably believed that extra anticholinergic function of diphenhydramine elevated the potency of the whole formula in sneeze control, stop, and management, as well as wellness maintenance of a nasal system.

### Example 25

On a day of early April 2014, the weather was cool and sunny. In the morning, the 53 year old chronic sneezer used the same anti-sneeze application as in Example 22, and then took the family for a whole day excursion. He did not sneeze at all during the driving, nor in the cool wind of the playground, nor in the warming sunshine, nor in the traffic fume, nor in the fumes and odors of factory outlet department stores. It was an amazing experience he had not had in past 10 or so cool spring seasons. He had suffered from spells of sneezing in past 10 years in the occasions such like this. He was glad that the suffering of unexpected, uncontrollable, and random sneezing would come to an end.

### Example 26

An anti-sneeze nose stick was made. A mixture of one part water, one part glycerin, one part propylene glycol, was added with 10 mg phenylephrine HCl, 10 mg cetirizine HCl, and 25 mg diphenhydramine HCl. The mixture then was compounded with one part of carrageenan. Both mint extract and lemon extract were added as aromas. The compounded mixture was a knead-able thick paste or dough. The total weight of the compounded mixture was 30 g. So phenylephrine, cetirizine, and diphenhydramine were 0.03%, 0.03%, and 0.083%, respectively, of the mixture. A 5 cm long coffee straw was cut to be used as a core, around which the mixture paste/dough was moulded into a stick. The final stick of 6 cm in length had a middle diameter of 1 cm. The stick tapers gradually from the middle to the two end tips. The rounded tip had a diameter of about 0.5 cm. The graduated stick of rounded tip fits nicely into an adult nose vestibule. It can be swirled around and against the nose vestibule skin for the purpose of anti-sneeze application. The aroma smells pleasantly. As such, a prototype of anti-sneeze nose stick was formed. It provides a basis for more elaborated manufacturing.

### Example 27

An anti-sneeze nose stick was made, which had an internal mechanism of wicking the solution to the tip for nasal vestibule application. The tapering part of a plastic pipette (dropper) was cut off. The remaining part had a length of about 7 cm. The bottom was filled with an anti-sneeze solution same as in Example 24 to a half filled level. Cotton fibers were stuffed into it. A section of 7.8 cm long coffee straw was inserted in all the way to the bottom. More cotton fibers were stuffed in and wrapped around the exposed coffee straw end. With lateral squeezing, the liquid wicked through fibers to the tip. The liquid was also seeping through the coffee straw to the tip at the squeezing. The formed device was tried on a subject with the cotton wrapped tip sticking into his nasal vestibules. It performed as expected when swirled around, massaged against his vestibule skin, and when the anti-sneeze liquid was applied to the skin. It was capped for storage and later use after the application. It is particularly convenient for carrying around and travelling. It is a new prototype of anti-sneeze nose stick. It can be more elaborated in a manufacturing process.

### Example 28

An anti-sneeze nose stick was made, which had an internal mechanism of wicking the solution to the tip for nasal vestibule application. A portion from the tapering end of a plastic pipette (dropper) was cut off. The remaining part had a length of about 7 cm. The bottom was filled with an anti-sneeze solution same as in Example 24 to a half filled level. In it, cotton fibers were stuffed to the full. Then the open end was sealed with a plastic cap using superglue. The round bottom was punctured with a fine needle in five positions. One was in the center, and the other four were around the center. The punctures served as capillary wicking channels when the device was squeezed laterally. As expected, at lateral squeezing, the anti-sneeze liquid contained in the fibers would ooze out through the punctures to wet the round bottom outer surface. In this case the wet round bottom would serve as a tip for nasal vestibule application of anti-sneeze formula to the skin. As such, a prototype of anti-sneeze device was established. It can be more elaborated in a manufacturing process.

### Example 29

An anti-sneeze nose stick/applicator was made, which had an internal mechanism of re-wetting the tip of the applicator for nasal vestibule application. A portion from the tapering end of a plastic pipette (dropper) was cut off. The remaining part had a length of about 4 cm. The bottom was filled with an anti-sneeze solution same as in Example 24 to a half filled level. In it, cotton fibers were stuffed to the full in a loose state. The liquid was fully absorbed by the fibers, the fibers were fully wet, but not dripping. The cut-off portion of the pipette was re-sized into a 3 cm tapering tube. A rod of 5 cm long cotton swab was inserted into the tube to the tapering end and superglue was applied and two parts were glued together. The cotton swab was then inserted into the pipette bottom in which the anti-sneeze composition was contained. The cotton swab was readily wetted with the formula for nasal vestibule application. After an application, the cotton swab would be inserted back and wetted again for next use. As such, a prototype of anti-sneeze device was constructed, and may be elaborated further in a manufacturing process.

### Example 30

On a day of middle April 2014, a 53 year old chronic male sneezer and a 53 year old female used the anti-sneeze application formula same as in Example 22 at 8 am. Before the anti-sneeze application, the female had a spell of sneezes in the early morning. They started to work on their yard to collect last fall leaves. The work took about 1 to 2 hours. The female had a sneeze at 11 am after the work, used the anti-sneeze application again, and remained sneeze-less for the rest of day. The male felt strong allergic reaction in his nose, face, and eyes. He realized that he forgot to use a face mask. The irritants in degraded fall leaves and yard debris caused an overwhelming allergic reaction. He took a pill of 10 mg cetirizine around 12 o'clock noon time. The oral drug did not take effect as expected. He suffered a severe running nose and sneezes. He used the anti-sneeze sticks and cotton swabs described in previous examples frequently. At each use, the application provided a temporary relief. The allergic reaction abated after 10 pm after he went to bed. Perhaps the oral drug took effect in his sleep, but it was the utilization of presently invented anti-sneeze composition and devices made his day time more manageable.

### Examples 31

A nose wellness care product was formulated. The formula contained 10 mg cetirizine HCl, 10 mg phenylephrine HCl, 25 mg diphenhydramine HCl, 0.06 g ascorbic acid (as antioxidant), and 0.06 g sorbic acid (as antimicrobial). The solvent of total 30 ml consisted of one part water, one part glycerin, one part propylene glycol, and one part aloe vera liquid extract (pure, as natural anti-inflammatory). At last, 2 g mint extract and 2 g lemon extract were added as aroma. The final formulated product was applied by two adult subjects to their nose vestibules with cotton swabs. The experience was described as pleasant, soothing, and relaxing to both nasal systems. From all the descriptions in aforementioned examples, it was understood that the application had anti-sneeze effect as well.

The foregoing descriptions of novel and example embodiments of the invention have been presented for purposes of illustration and description. The descriptions are not intended to be exhaustive or to limit the invention to the precise form disclosed. Obvious modifications or variations are possible in light of the above testing. The embodiment was chosen and described to provide the best illustration of the principles of the invention and its practical applications to thereby enable one of ordinary skill in the art to utilize the invention in various embodiments and with various modifications as are suited to the particular use contemplated. All such modifications and variations are within the scope of the invention as determined by the claims made in this application when interpreted in accordance with the breadth to which they are fairly, legally and equitably entitled.

What is claimed is:

1. An anti-sneeze or anti-sneezing method for stopping, reducing, controlling, or managing sneezes consisting topically and manually applying a composition to the skin of nasal vestibules of a subject, wherein the composition contains essentially an adrenergic agent, an antihistamine agent, an anticholinergic agent, a plasticizer for the skin and muscle of the nose, and a soothing agent.

2. A nose vestibule care method, in the category of personal care or animal care methodology, consisting of topically and manually applying a composition to the skin of nasal vestibules of a subject, wherein the composition contains essentially an adrenergic agent, an antihistamine agent, an anticholinergic agent, a plasticizer for the skin and muscle of the nose, a soothing agent, plus an optional anti-inflammatory agent, an optional antimicrobial agent, and an optional antioxidant agent.

3. A medicated device that is to be used as a tool to deliver an anti-sneeze composition or a wellness care composition to the skin of the nose vestibules of a subject for the purpose of stopping, reducing, control, and management of sneezes, or for the purpose of preventive care against air-born disease causing species, pollutants, and irritants, or for the purpose of aiding a medical treatment before the medical treatment, between the medical treatments, and after the medical treatment.

4. The adrenergic agent of claim 1 is for purposes of constricting, or contracting nose vestibule skin and muscle.

5. The plasticizer agent of claim 1 is for relieving the rigidity caused by the constriction in nose vestibule skin and muscle.

6. The soothing agent of claim 1 further counteracts and neutralizes any discomfort caused by the constriction in nose vestibule skin and muscle, and provides a pleasant induction of the composition to the nose vestibules.

7. The anti-inflammatory agent of claim 2 is for preventive care of/or reducing infections that are common in the base of the hairs within the vestibule.

8. The anti-cholinergic agent of claim 1 is for preventive care of/or reducing excessive secretion of the nose through interaction with the nerves in the lining of the nose vestibule.

9. The antihistamine agent of claim 1 is for suppressing the histamine-induced wheal response (swelling) and flare response (vasodilation) in the lining of nose vestibules, in the spirit of prevention of hay fever, itching and rashes in case of presence of air-born allergens or irritants.

10. The antimicrobial agent of claim 2 is for prevention or inhibition of air-born or contact transmitted microbial (virus, bacterial, fungus, yeast, and parasite) infection in the lining of nose vestibules.

11. The antioxidant agent of claim 2 is for reduction of harmful oxidant molecules from air pollutants in the lining of nose vestibules.

12. The medicated device of claim 3 is a cotton swab, or other type of absorbent fiber swab, or an absorbent polymer matrix, that is impregnated with aforementioned anti-sneeze composition/or nose vestibule care composition. The swabs are packaged and sealed either individually, or in groups, for storage, distribution, sale, and end consumer use.

13. The medicated device of claim 3 is a “nose stick” tube, similar to a lipstick style tube. The aforementioned anti-sneeze composition/or nose vestibule care composition is compounded in a solid waxy/polymeric material so it can be spread easily but remains stiff as a core in the tube. The stick has a cone shaped tip or any other gradually tapered shape with smooth tip, which is pressed, swirled and massaged against the lining of a vestibule to deliver the composition.

14. The medicated device of claim 3 is a “nose stick” tube, similar to a lipstick style tube. The inside body of the tube is a liquid containing solid absorbent material, or a foam. The aforementioned anti-sneeze composition/or nose vestibule care composition is retained in the liquid of the tube. The composition is readily applied to the lining of a nose vestibule when the cone shaped tip or a smooth tip of any shape of the solid absorbent material or foam is pressed, swirled and massaged against the lining. The composition liquid can wick through the absorbent tube to the tip, or flow through to the tip by a pressure applied from the bottom, or by a lateral squeeze.

15. The medicated device of claim 3 is a “nose stick” tube. The tube is a liquid container with a cone-shaped tip or a smooth tip of any shape. The tip has capillaries of liquid passage. The contained liquid in the tube comprises the aforementioned anti-sneeze composition/or nose vestibule care composition. When the tube is squeezed or applied with a pressure, the liquid is readily wicked through the capillaries to the surface of the tip, which is pressed, swirled and massages against the lining of the vestibule to deliver the composition.

16. For a liquid composition in claim 1, the solvent can be either demineralized water, or a non-aqueous medium.

17. The medicated device of claim 3 is a “nose stick” tube, similar to a lipstick style tube. The cap of the tube is stuffed with a solid absorbent material, or foam. The aforementioned anti-sneeze composition/or nose vestibule care composition is retained in the absorbent design. The tapering tip of the stick has an absorbent outer layer, and is wetted by the liquid composition in the cap when it is encased. The liquid composition is readily applied to the lining of a nose vestibule when the smooth tip of the stick is pressed, swirled and massaged against the lining. The tip is wetted again when it is re-capped.

18. The liquid composition according to claim 16, further including a preservative, a viscosity adjuster, a tonicity modifier, a dye or pigment for colored appearance.

19. The liquid composition according to claim 17, further including a preservative, a viscosity adjuster, and a tonicity modifier, a dye or pigment for colored appearance.

20. Any of the agents in claim 2 can be either synthetic, or of a natural origin, like from a botanical extract, herbal extract, or a sea plant extract.
